(19) United States
(12) Patent Application Publication
(10) Pub. No.: US 2006/0149211 A1
(43) Pub. Date: Jul. 6, 2006

(54) Controlled flow apparatus for medical accessories

(75) Inventors: Philip J. Simpson, Escondido, CA (US); David G. Matsuura, Encinitas, CA (US); Walter Dean Gillespie, San Diego, CA (US); James A. Trinchera, Encinitas, CA (US)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: VASOGEN IRELAND LIMITED, County Clare (IE)

(21) Appl. No.: 11/312,584

(22) Filed: Dec. 21, 2005

Related U.S. Application Data
(60) Provisional application No. 60/640,088, filed on Dec. 30, 2004.

(51) Int. Cl. A61B 19/00

(52) U.S. Cl. 604/403; 604/246

(57) ABSTRACT

An apparatus for controlled flow of fluid materials for use with medical accessories has a first housing with a body, a first open end, a second open end, and a hollow interior. The second end fits with a first medical accessory for flow of fluid materials from the first medical accessory. A fluid flow control means is disposed within the housing for controlling the flow of fluid materials through the hollow interior from the first medical accessory. The apparatus also has a second housing with an open end, a first open end, a hollow interior, and a second open end having an axial bore coaxial with the hollow interior of the first housing. The second end of the second housing fits with the first open end of the first housing. The first open end of the second housing fits with a second medical accessory, whereby the second medical accessory receives fluid materials from the first medical accessory by way of the hollow interior of the body.

This application is based on and claims priority under 35 U.S.C. §119(e) with respect to U.S. provisional Application No. 60/640,088 filed on Dec. 30, 2004, the entire content of which is incorporated herein by reference.

### FIELD OF THE INVENTION

The present invention relates to an apparatus for use with medical accessories for transfer of fluid materials, and more particularly a controlled flow apparatus.

### BACKGROUND ART

There are many instances in hospitals or in medical situations, where there is the need to transfer fluids from one source or medical accessory to a recipient, receptacle, or second medical accessory. Often, these fluids could be toxic or could pose contamination hazards. Accordingly, contamination of the environment and possible exposure of patients and medical professionals alike with biological fluids or medicines is a serious issue for medical professionals.

For example, blood is commonly drawn from a patient using a butterfly needle. However, since using a cap at the end of the tubing for a butterfly needle is not convenient, most nurses or health professionals will merely crimp the tubing to prevent blood flow from the patient to the end of the tubing. The health professional then attaches the tube to the appropriate receptacle to allow the transfer of blood from the patient to the receptacle. This method of controlling the flow and transfer of blood from a patient is cumbersome and prone to human error.

In another example, a chemotherapy agent may be administered to a patient. However, most chemotherapy agents can be toxic with concomitant dangers to the patient, medical staff, and the environment if there were an accidental release of the agent. There is a need for an apparatus that minimizes the risk of escape of the chemotherapy agent prior to infusion so as to prevent contamination of the environment or exposure of the patient or health professional to a potentially toxic compound.

In view of the foregoing, there is a need to have a controlled flow apparatus for fluid materials for use with medical accessories that reduces the risk of exposure of the environment, patient, or health professional to potentially hazardous fluids. Accordingly, it is an object of the present invention to provide a novel controlled flow apparatus for use with medical accessories. Medical accessories encompass syringes, containers, tubing associated with needles, or other apparatus that hold or transfer fluid materials in a medical setting. Fluid materials can encompass liquid and gaseous materials.

### SUMMARY OF THE INVENTION

According to one embodiment of this invention, a controlled flow apparatus for medical accessories is provided, comprising a first housing having a body, a first open end, a second open end, and a hollow interior. The second open end is dimensioned for fit with a first medical accessory for flow of fluid materials from the medical accessory. A fluid flow control means is disposed within the first housing for controlling flow of fluid materials through the hollow interior from the first medical accessory. As well, there is a second housing having a body, a first open end, a hollow interior, and a second open end dimensioned for fit with the first open end of the first housing. The second open end of the second housing has an axial bore coaxial with the hollow interior of the first housing. The first open end of the second housing is dimensioned for fit with a second medical accessory for receipt of fluid materials by the second medical accessory whereby there is flow of fluid materials into said second medical accessory from the first medical accessory, via the hollow interior of the body.

In another aspect of the invention, a controlled flow apparatus for medical accessories is provided having a first housing with a body, a first open end, a second open end, and a hollow interior, where the second open end is dimensioned for fit with a first medical accessory. A one-way valve is disposed within the first housing for controlling flow of fluid materials from the first medical accessory. Suitably, the one-way valve can be a duckbill valve or an umbrella valve. There is also a second housing where one end is dimensioned for fit with the first open end of the first housing and the other end of the second housing is dimensioned for fit with a second medical accessory. Preferably this embodiment includes a valve means in the form of a valve disposed within the first housing for controlling flow of fluid materials to and from the medical accessories. In another aspect, the valve disposed within the first housing for controlling flow of fluid materials to and from the medical accessories is a combination valve with a duckbill portion and an umbrella portion. In yet another aspect, the said valve comprises least two one-way valves, where at least one one-way valve permits fluid flow in an opposite direction from at least one other one-way valve.

In another aspect of the invention, a controlled flow apparatus is provided having a first housing with a first circumferential side wall, a circumferential bottom shoulder attached thereto, and a second circumferential side wall depending from the shoulder. The second circumferential side wall has an inner diameter smaller than the inner diameter of said shoulder and defines an axial bore. This embodiment has similar structure with respect to the second housing and fluid flow control means as the prior embodiment and functions in essentially the same way as the prior embodiment.

In further aspect of the invention, a controlled flow apparatus is provided having a first housing with an end dimensioned for fit with a first medical accessory, fluid flow control means disposed within the first housing, a second housing having an end dimensioned for fit with the other end of the first housing, wherein the second housing has a circumferential side wall, a bottom plate attached thereto, an aperture in the bottom plate, and a hollow needle disposed within the aperture for fluid communication with the valve means. The circumferential side wall is dimensioned for fit with a second medical accessory such as a receptacle or syringe with a septum that is pierceable by the hollow needle to permit fluid flow, under the control of the second medical accessory, from the first medical accessory to the second medical accessory. In another aspect of the invention as described in the above embodiments, the valve means is actuable by the second medical accessory. In yet another aspect, the second housing comprises a circumferential wall extending axially outwardly from the body and a shoulder depending radially, inwardly from the end of the second housing proximal to the body and defining an aperture. Preferably the second housing has retaining means to retain a medical accessory in association with the second housing. In a further aspect, actuating means are associated with the second housing to actuate a medical accessory attached to the second housing to provide fluid communication between the medical accessory and the controlled flow apparatus.

Suitably, the ends of the first and second housings that are dimensioned for fit with medical accessories are luer fittings.

This invention also provides a controlled flow apparatus having a body with a hollow interior and two ends. A first housing extends from one end of the body, is in fluid communication with the hollow interior, and has an end distal to the body which is dimensioned for attachment with a first medical accessory. A first fluid flow control means is disposed within the first housing for controlled one-way flow of fluid materials into the interior of the body from the first medical accessory. A second housing depends from the other end of the body and is in fluid communication with the hollow interior. The end of the outlet housing distal to the body is dimensioned for fit with a second medical accessory. A second fluid flow control means is disposed within the outlet housing for controlled flow of fluid materials from the interior of the body to the second medical accessory. In yet another aspect of the invention, the first fluid control means controls fluid flow to and from the first medical accessory. The first fluid control means controlling fluid flow to and from the first medical accessory may encompass a valve controlling flow of fluid materials to and from the first medical accessory, a combination valve permitting fluid flow to and from the first medical accessory, or may comprise at least two one-way valves, wherein at least one one-way valve permits fluid flow in an opposite direction from at least one other one-way valve. In a further aspect of the invention, the second fluid flow control means controls flow of fluid to and from the second medical accessory. The second fluid control means controlling fluid flow to and from the first medical accessory may encompass a valve controlling flow of fluid materials to and from the second medical accessory, a combination valve permitting fluid flow to and from the second medical accessory, or can comprise at least two one-way valves, where at least one one-way valve permits fluid flow in an opposite direction from at least one other one-way valve.

Preferably the controlled flow apparatus is described of the invention has an additional outlet depending from the body, in fluid communication with the hollow interior. A gas flow control means can be positioned within the additional outlet to permit one-way flow of gaseous material, without expulsion of liquid material, out of the interior of the body. A hydrophobic filter is positioned in association with the aperture at the end of the additional outlet proximal to the interior of the body, to prevent liquid from entering the housing and being expelled from the outlet. Preferably, an oleophobic membrane is associated with the hydrophobic filter to prevent the hydrophobic filter from being clogged with components of the fluid material that would lessen the efficiency of the hydrophobic filter.

In another aspect of this embodiment of the invention, the second housing comprises a circumferential wall extending axially outwardly from the body and a shoulder depending radially, inwardly from the end of the second housing proximal to the body and defining an aperture. The second fluid flow control means is positioned within the second housing and is biased against the shoulder for a fluid tight fit. A valve seat is preferably attached to the shoulder and defines an aperture, and the second fluid flow control means includes a valve head which abuts against the valve seat to create a fluid tight seal. The second fluid flow means may further include a nose portion that extends axially outwardly from the bottom of the second housing proximal to the interior of the body. The valve seat is suitably made of elastomeric material to assist in creating a face seal with the second medical accessory to prevent leakage of fluid materials when the second medical accessory is mounted in the outlet housing. The valve seat preferably has an upstanding annular ring that assists in creating a face seal for fluid tight fit with the second medical accessory.

Preferably also, the outlet housing has retaining means to retain the second medical accessory in association with the outlet housing. The second valve means may be actuatable by the second medical accessory.

### BRIEF DESCRIPTION OF THE DRAWINGS

FIG. 1 is an exploded perspective view of a controlled flow apparatus for medical accessories according to a first preferred embodiment of the invention.

FIG. 2 is a longitudinal cross-sectional view of the apparatus of FIG. 1;

FIG. 3 is a partial cross-sectional view of the apparatus of FIG. 1 where a syringe is mounted on one end;

FIGS. 4 and 4a are perspective views of a second preferred embodiment of a controlled flow apparatus for medical accessories according to the invention;

FIG. 5 is an underneath plan view of the second preferred embodiment illustrated in FIG. 4;

FIG. 6 is a top plan view of the second preferred embodiment of the invention illustrated in FIG. 4;

FIG. 7 is a front view of the embodiment illustrated in FIG. 4;

FIG. 8 is a back view of the embodiment illustrated in FIG. 4;

FIG. 9 is a cross-sectional view along the line 9-9 of FIG. 4a;

FIG. 10 is a cross-sectional view along the line 10-10 of FIG. 4a;

FIG. 11 is a cross-sectional view along the line 11-11 of FIG. 4a; and

FIG. 12 is a cross-sectional view along the line 12-12 of FIG. 4a.

FIG. 13 is a perspective view of the third preferred embodiment of the invention.

FIG. 14 is a side view of the embodiment of the invention illustrated in FIG. 13.

FIG. 15 is a front view of the embodiment of the invention illustrated in FIG. 13.

FIG. 16 is a cross-sectional view along the line A-A of FIG. 15.

### DESCRIPTION OF THE PREFERRED EMBODIMENTS

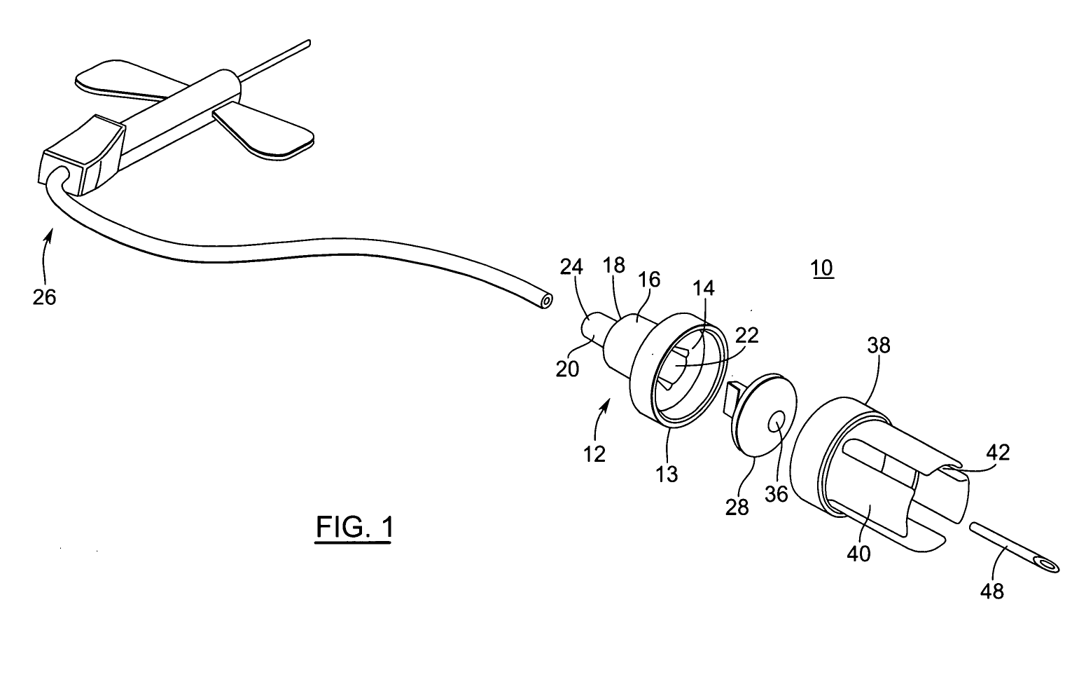

FIG. 1 shows generally a controlled flow apparatus of one of the preferred embodiments of the present invention. Controlled flow apparatus 10 comprises a housing 12 having a first circumferential side wall 13, a first annular end wall 14, a second circumferential side wall 16 extending therefrom having an inner diameter less than the inner diameter of first circumferential side wall 13, a second annular end wall 18 extending therefrom and defining an axially extending shoulder, and a third circumferential side wall 20 having an inner diameter less than that of the second circumferential side wall 16. The housing 12 defies a central bore 22 coaxial to the second circumferential side wall 16 and third circumferential side wall 20. Distal end 24 of third circumferential side wall 20 is adapted to fit with a medical accessory, in this case a butterfly needle with medical tubing 26.

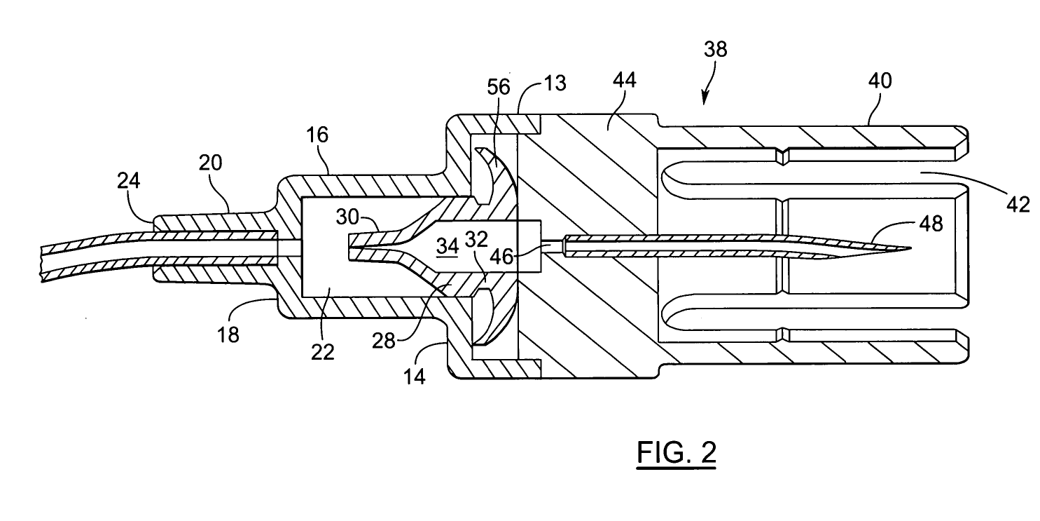

Disposed within central bore 22 is a combination valve 28 (FIG. 2) which permits independently controlled flow in both directions from butterfly needle 26. Combination valve 28 has a duckbill valve portion 30, an umbrella valve portion 32, and an interior space 34 that is in fluid communication with the end of umbrella valve portion 32 by way of aperture 36.

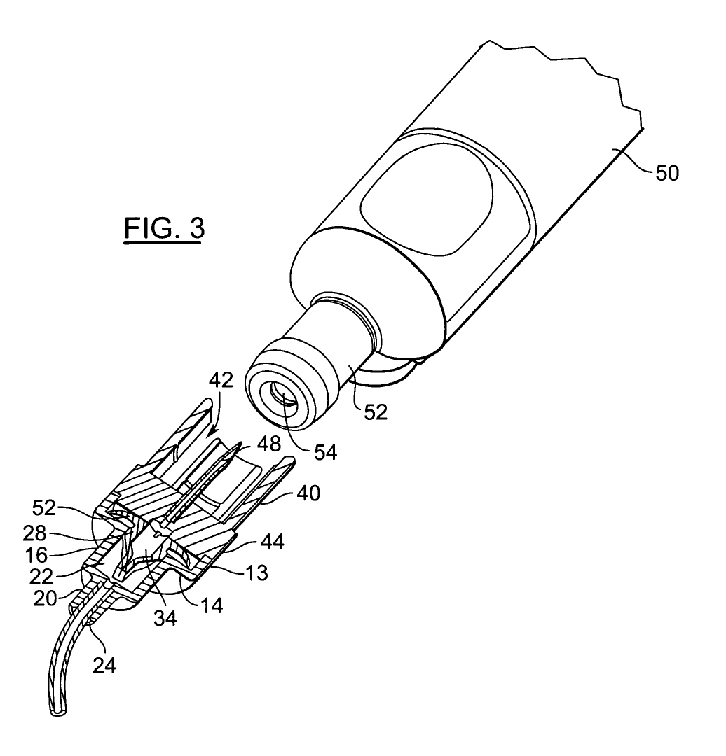

An end housing 38 is provided, dimensioned for fit with the proximal end of lower housing 12. End housing 38 has upstanding side walls 40 (FIG. 3), separated by a plurality of slots 42. End housing 38 has a circumferential bottom plate 44 with a central passageway 46 for fluid communication with aperture 36 of combination valve 28. A needle 48 is disposed in passageway 46. Fluid from butterfly needle 26 cannot flow through combination valve 28 when the umbrella portion 32 is closed.

As seen in FIG. 3, a syringe 50 having a front outlet 52 with a sealable septum 54 is mounted on the front of end housing 38 such that needle 48 pierces septum 54 to allow for fluid communication with the interior of the syringe 50. When plunger (not shown) of syringe 50 is pulled back, a vacuum is created which causes arms 56 of umbrella valve portion 32 to pull back and allow fluid flow around combination valve 28 and into syringe 50. When the plunger is depressed, fluid from syringe 50 is pressurized and passes through septum 54 and needle 48. Assuming the pressure is greater than the cracking pressure of duckbill valve portion 30, this will open to allow fluid to pass through combination valve 28, in the direction of distal end 24.

Although housing 12 and end housing 38 have been described as being separate parts, they can also be integrally formed. Even though combination valve 28 having a duckbill and umbrella valve portions has been described as a preferred embodiment, any suitable fluid flow control means can be used such as means for two or one way flow (i.e. duckbill valve, umbrella valve), including combinations of one way flow means. It is also to be appreciated that although end housing 38 has upstanding side walls 40 interspersed with slots 42, end housing 38 can have an upstanding, continuous, circumferential side wall.

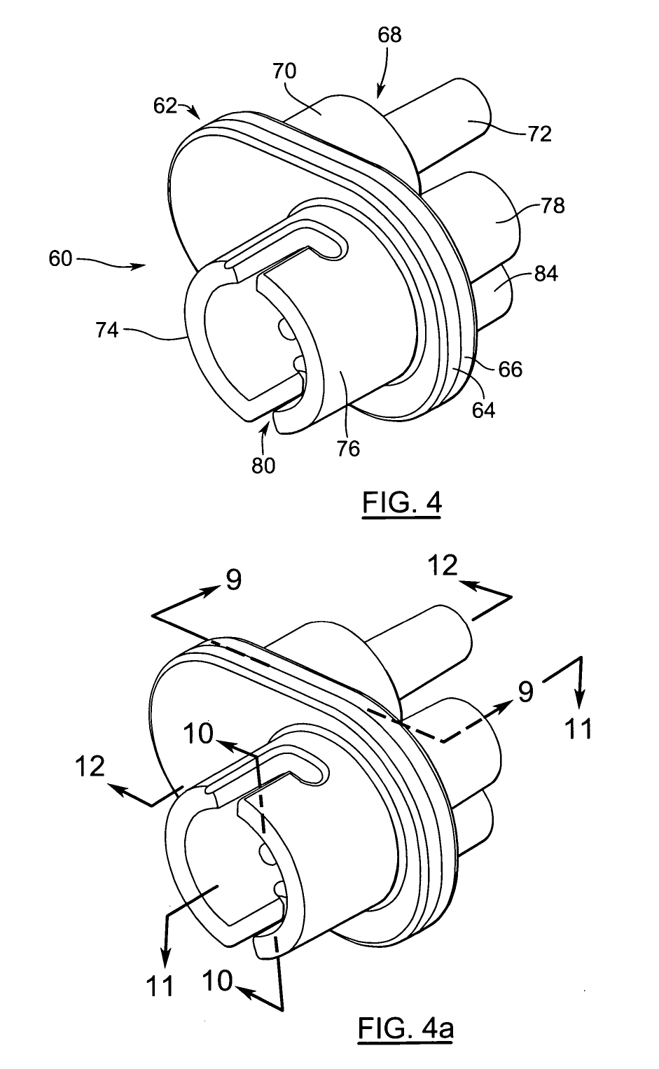

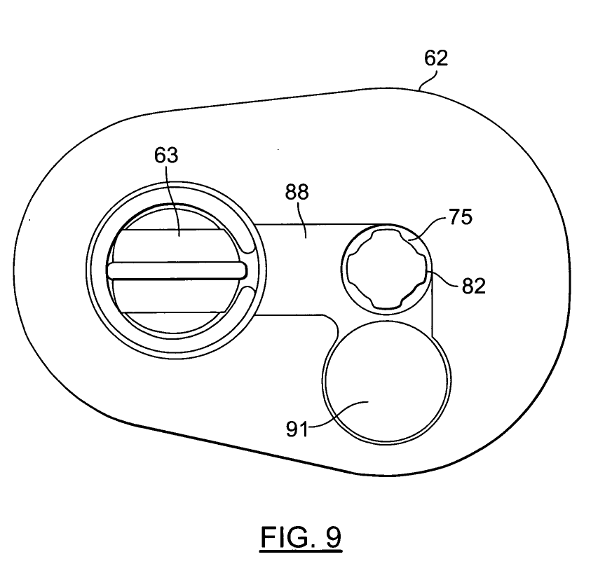

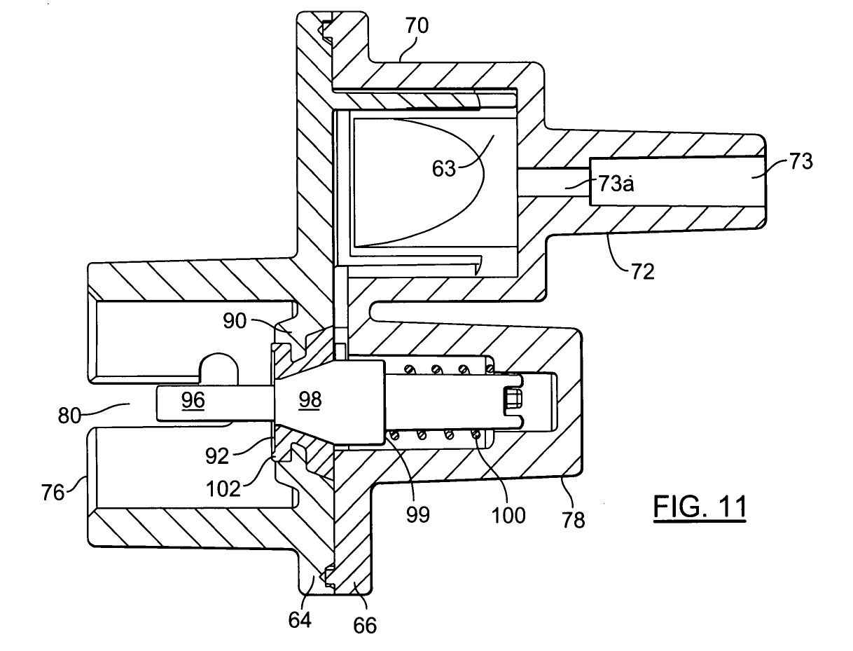

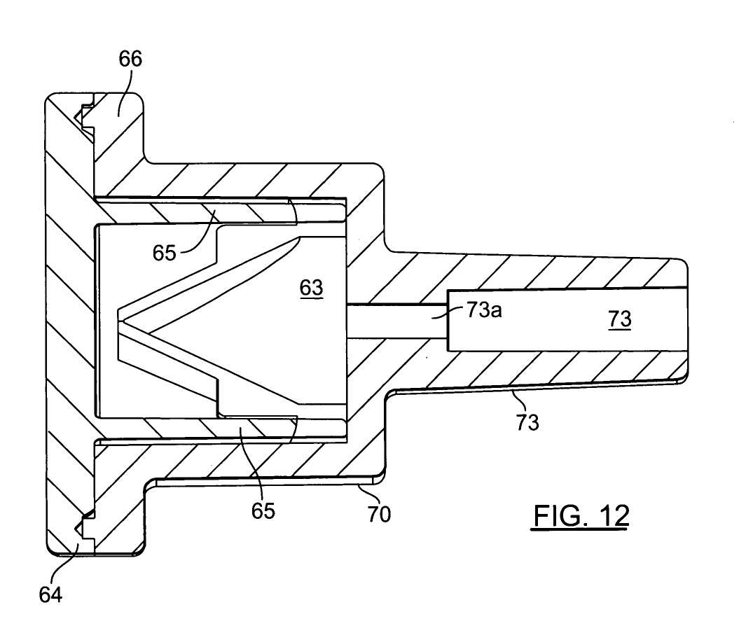

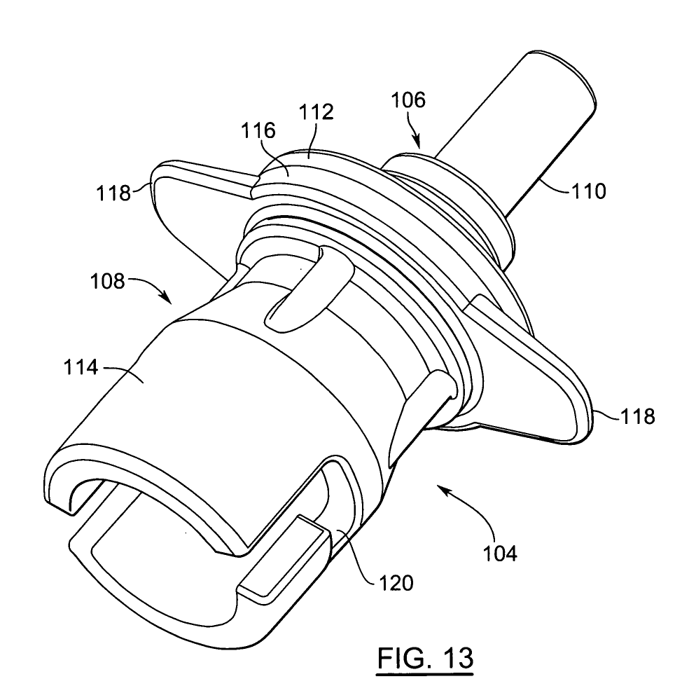

FIGS. 4-12 illustrate another preferred embodiment of the invention. This controlled flow apparatus 60 comprises a body 62 that is composed of parts 64 and 66. There is an inlet housing 68 extending outwardly from part 66 with a first circumferential wall 70, and a second circumferential wall 72 having a smaller outer diameter than first circumferential wall 70. The second circumferential wall 72 defines an axial bore 73 (FIGS. 11 and 12). Axial bore 73 is coaxial with a passageway 73a. The end of second circumferential wall 72 is dimensioned for fit with a medical accessory (not shown) to permit controlled fluid material flow between the medical accessory and the interior of body 62. A duckbill valve 63 (see FIGS. 9, 11 and 12) is positioned within first circumferential wall 70 to control the flow of fluid materials from the medical accessory attached to the end of wall 72. There is fluid communication between the end of circumferential wall 72 and the interior of duckbill valve 63 (see FIG. 11) by way of axial bore 73 and passageway 73a. A circumferential wall 65 (FIG. 12) extends from part 64 and has an outer diameter slightly less than the inner diameter of first circumferential wall 70 for snug fit. The end of circumferential wall 65 distal to part 64 serves to hold duckbill valve 63 in place.

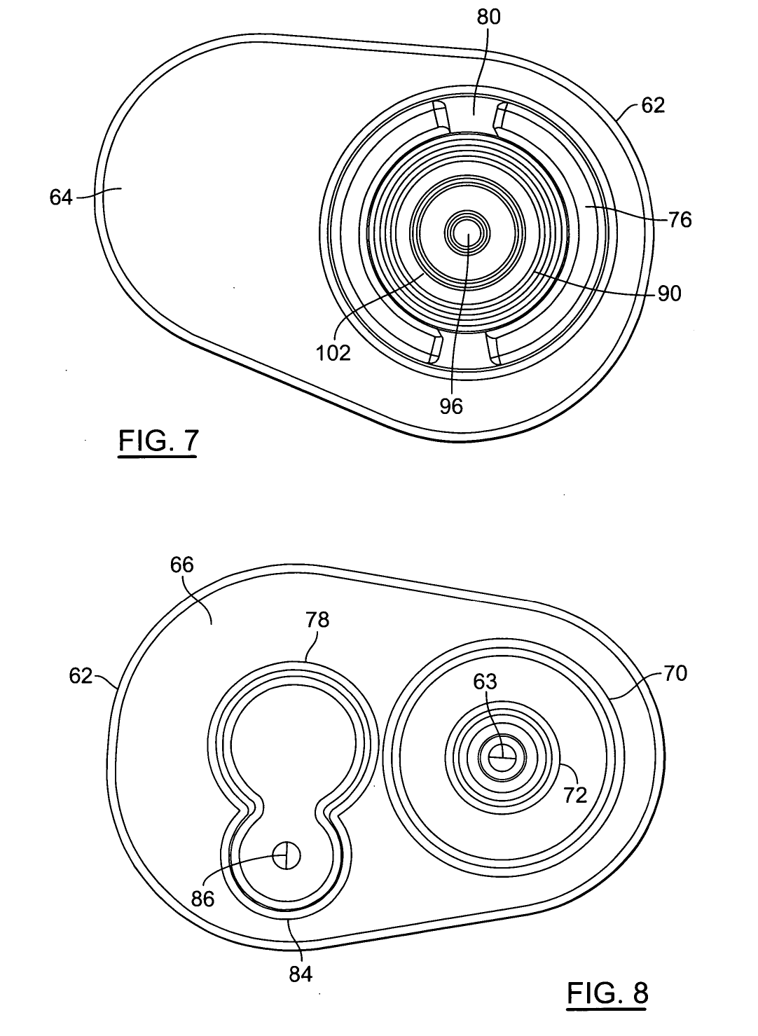

There is an outlet housing 74 extending outwardly from part 64 (FIG. 4) and also extending outwardly from part 66. Outlet housing 74 has a circumferential wall 76 extending outwardly from part 64 and has a circumferential wall 78 extending in the opposite direction from part 66 and having an outer diameter smaller than the outer diameter of circumferential wall 76. Circumferential wall 76 has bayonet slots 80 to assist in retaining a medical accessory having corresponding bayonet pins (not shown). A valve 82 (FIGS. 6 and 9) is disposed within outlet housing 74 to permit controlled flow of fluid materials from the interior of body 62 into the medical accessory retained within outlet housing 74.

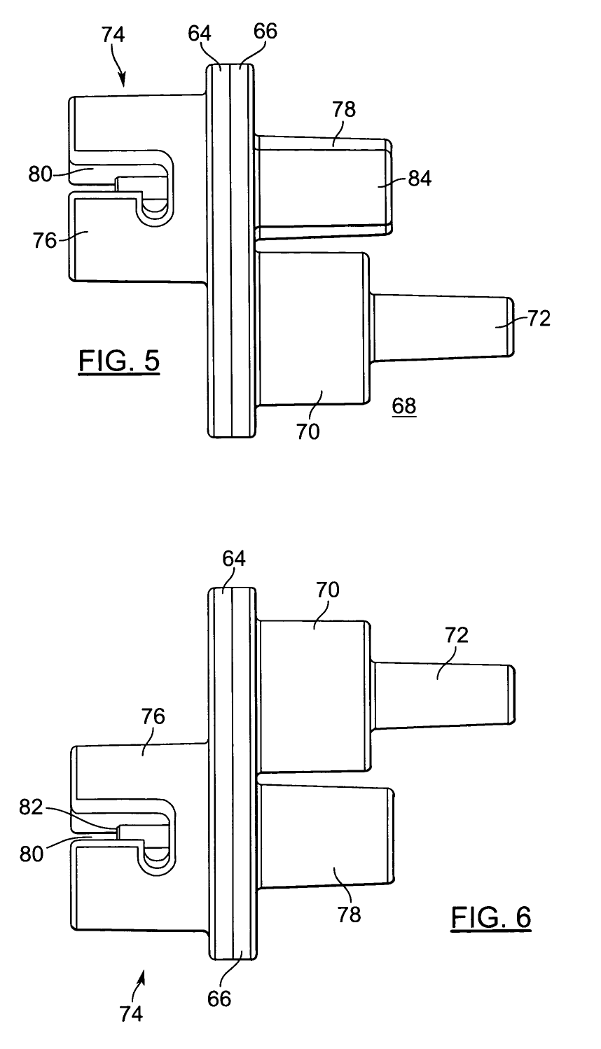

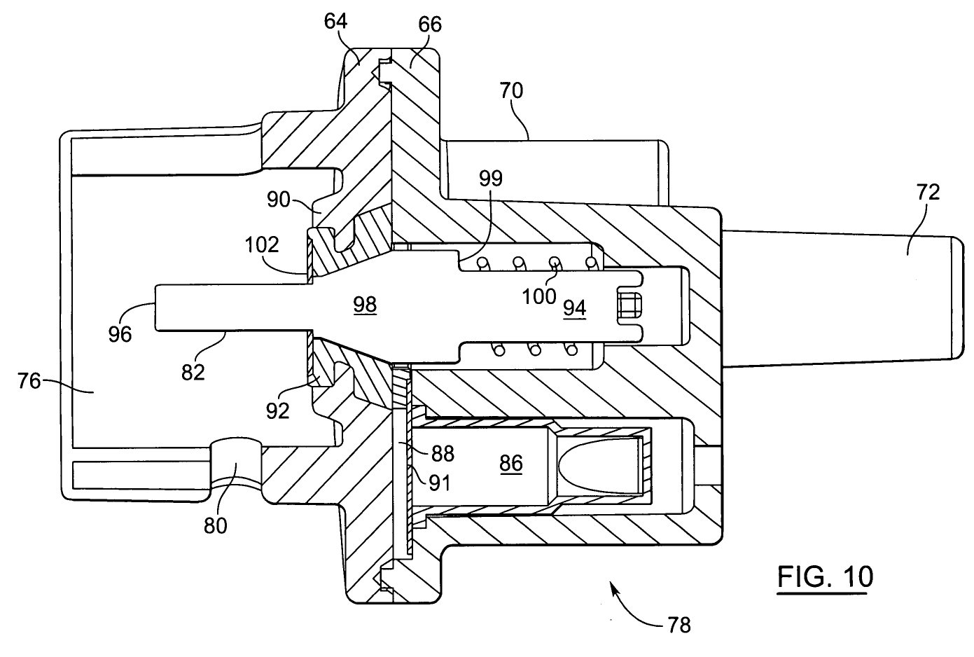

This embodiment includes a gas outlet housing 84 (FIGS. 4 and 5) extending from part 66 and is, in part, formed with circumferential wall 78. Duckbill valve 86 (see FIG. 10) positioned within gas outlet housing 84 permits one-way expression of gas from the interior of body 62 to the environment, thereby lessening the likelihood of transfer of gases along with fluid material, from the interior of body 62 or from the medical accessory which transfers fluid materials into body 62, by way of inlet housing 70, to the medical accessory attached to outlet housing 74. As well, the likelihood of contamination of the fluids by gases entering into interior 88 through gas outlet housing 84 is minimized because of duckbill valve 86.

In body 62 there is a hollow interior 88 (see FIGS. 4 and 9) that permits fluid communication between inlet housing 68, outlet housing 74, and gas outlet housing 84. A hydrophobic membrane 91 covers the opening of gas outlet housing 84 proximal to interior 88 such that gas, and not liquid materials, may pass from interior 88 through gas outlet housing 84 to the environment, thus allowing gases to be purged from interior 88 and lessening the likelihood that gases will be transferred to the medical accessory fitted in the outlet housing.

As seen in FIGS. 9 and 10, interior 88 is in fluid communication with the interior 75 of outlet housing 74. Circumferential wall 76 has a radially inwardly extending circumferential shoulder 90 to which is attached valve seat 92. Valve 82 consists of valve body 94 having a nose 96, a valve head 98, and radially inwardly extending shoulder 99 which serves to abut against one end of a resilient spring 100. In its normal position, valve head 98 is biased against valve seat 92 by spring 100 for fluid tight fit and thus prevent flow of liquid from interior 88 through outlet housing 74. When a medical accessory is fitted to outlet housing 74, the medical accessory has actuating means to push nose 96 backwards to unseat valve head 98 from valve seat 92, thus permitting fluid materials to flow from interior 88. The medical accessory has a surface proximal to the outer surface of shoulder 98 and valve seat 92 that forms a face seal with an annular ring 102 to create a fluid tight fit so as to prevent leakage of fluid between the medical accessory and outlet housing 74.

One example of an application for controlled flow apparatus 60 is for controlled flow of blood from a patient. One end of medical tubing is connected to circumferential wall 72 while the other end is attached to a butterfly needle. When the butterfly needle is inserted into a patient's vein, the blood flashes up the medical tubing under the venous pressure of the patient. The cracking pressure of one way duckbill valve 63 is less than the venous pressure of the patient, thus permitting blood to pass through the medical tubing, inlet housing 68, and duckbill valve 63 into interior 88, without the risk of blood going backwards through duckbill valve 63 to the patient.

Air within the medical tubing and interior 88 is purged from interior 88 through gas outlet housing 84. Since the cracking pressure of one way duckbill valve 86 is less than the venous pressure of the patient, when blood flashes up into interior 88, most of the air from the medical tubing and interior 88 is purged through gas outlet housing 84. Hydrophobic membrane 90 allows air to pass through duckbill valve 86 and gas outlet housing 84 into the environment without permitting blood to pass through gas outlet housing 84, thus maintaining sterility of the blood. Given that the venous pressure of the patient is sufficient to drive blood up through the medical tubing, through inlet housing 68 (by way of duckbill valve 63), into interior 88, the tubing and interior 88 are essentially purged of gases so as to reduce the likelihood that gases will be transferred through outlet housing 74, along with blood, to the medical accessory fitted with outlet housing 74.

Meanwhile, blood cannot exit controlled flow apparatus 60 once it enters into interior 88 unless valve 82 is actuated. Owing to the presence of the one way duckbill valve in inlet housing 68, blood cannot go back to the patient after having entered interior 88. In addition, blood cannot exit through gas outlet housing 84 because hydrophobic membrane 90 covers the open end of gas outlet housing 84 proximal to interior 88 and prevents blood from exiting gas outlet housing 84 to the environment, although gas can be purged from interior 88 through gas outlet housing 84.

A syringe, with a valve actuating means at the inlet end proximal to outlet housing 74 can fit onto outlet housing 74 and be retained in place with bayonet slots fitting within bayonet slots 80. When the syringe is held in place, the actuating means will push back valve body 94 rearwardly by the actuating means contacting nose 96. Valve head 98 is displaced from valve seat 92 and allows fluid communication between interior 88 and the interior of the syringe. In addition, the distal portion of the inlet end of the syringe can make a face seal with the outer surface of shoulder 90 and valve seat 92 to make a fluid tight fit between outlet housing 74 and the syringe to minimize the likelihood of blood escaping into the environment when the syringe is mounted on outlet housing 74. The syringe plunger can be pulled back to draw blood into the interior of the syringe body.

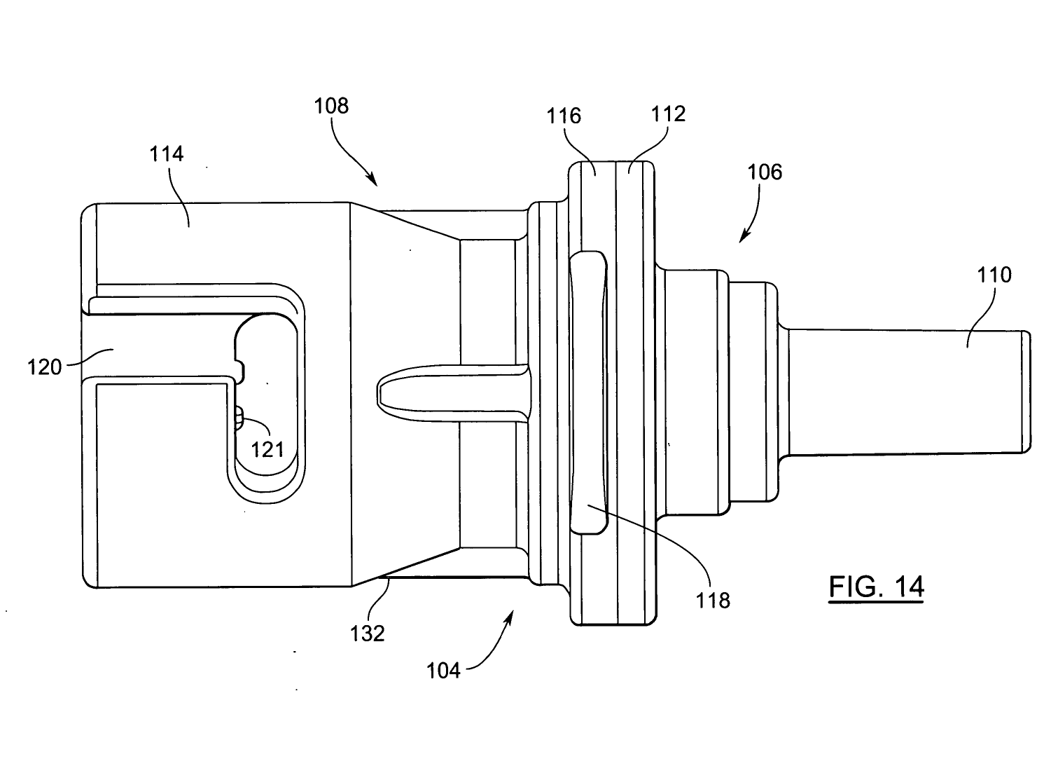

FIGS. 13-16 illustrate another preferred embodiment of the invention. Controlled flow apparatus 104 (see FIGS. 13 and 14) has an inlet housing 106 and an outlet housing 108. Inlet housing 106 is comprised of an inlet end 110 having a circumferential wall attached to the main body 112. Inlet end 110 has an outer diameter smaller than main body 112 and the end of inlet end 110 distal to main body 112 is dimensioned for fit with a medical accessory (not shown) for fluid flow between the medical accessory and inlet end 110.

Outlet housing 108 has a circumferential wall 114 extending axially from a main body 116 of outlet housing 108, where main body 116 is proximal to inlet housing 106 and attaches to main body 112 of inlet housing 106. The end of circumferential wall 114 distal to main body 116 is open. Main body 116 also has two extending wings 118. Outlet housing 108 is dimensioned for fit with a medical accessory (not shown) to permit fluid flow between the medical accessory and outlet housing 108.

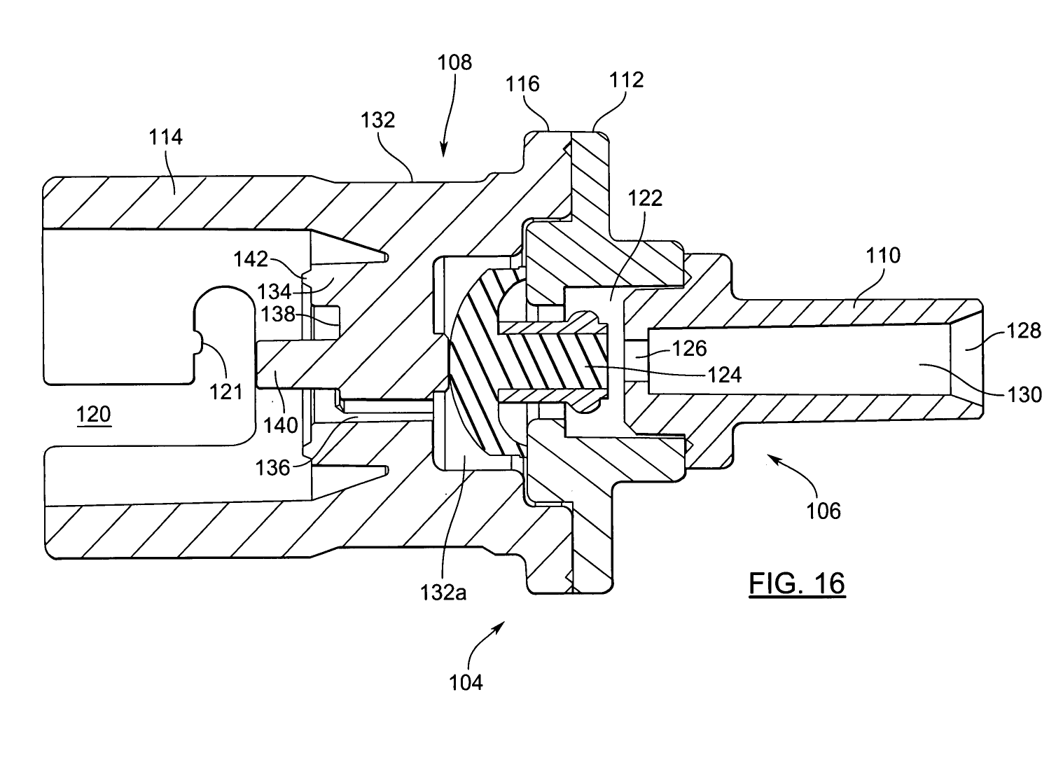

Circumferential wall 114 has bayonet slots 120 to assist in retaining a medical accessory having corresponding bayonet pins (not shown). Beads 121 (see FIGS. 14 and 16) assist in retaining bayonet pins within bayonet slots 120. Outlet housing 108, inlet housing 106, and inlet end 110 are attached together for fluid tight fit.

Main body 112 of inlet housing 106 has a hollow interior 122 (see FIG. 16) within which umbrella valve 124 is placed to control fluid flow into fluid control apparatus 104 and prevents fluid from flowing back into and out of inlet end 110. Umbrella valve 124 is held in place when inlet end 110, inlet housing 106, and outlet housing 108 are attached together. Inlet end 110 has open ends 126 and 128 and a hollow interior 130 such that there is fluid communication between inlet end 110 and main body 112.

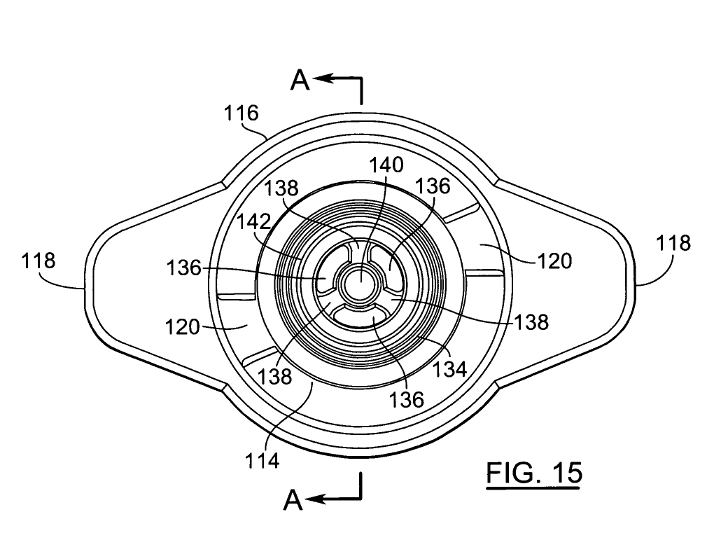

End 132 of circumferential wall 114 proximal to inlet housing 106 has a radially, inwardly depending shoulder 134 (see FIGS. 15 and 16), and has spaced apertures 136 coaxial with hollow interiors 122 and 130 to permit fluid communication between outlet housing 108 and inlet housing 106. Apertures 136 are bordered by arms 138 which extend radially inward from shoulder 134 (see FIG. 15). End 132 also defines hollow interior 132a. Arms 138 are attached at the center by nose 140 which extends axially outwardly, coaxial to the central axis of outlet housing 108, in the direction of the end of circumferential wall 114 distal to inlet housing 106 and serves to actuate a medical accessory (not shown) attached to outlet housing 108 such that there is fluid communication between the medical accessory and hollow interior 132a. Hollow interior 132a is coaxial with hollow interiors 122 and 130.

Shoulder 134 also has an upstanding annular ring 142 (see FIGS. 15 and 16). The medical accessory (not shown) that attaches to outlet housing 108 has a surface proximal to the outer surface of shoulder 134 that forms a face seal with annular ring 142 to create a fluid-tight fit so as to prevent leakage of fluid between the medical accessory and outlet housing 108. When the medical accessory is attached to outlet housing 108 with fluid-tight fit, the medical accessory can create the vacuum necessary to exceed the cracking pressure of duckbill valve 124, thus drawing fluid from the medical accessory (not shown) attached to inlet end 110, via hollow interiors 122, 130, and 132a, and apertures 136, without leakage of the fluid.

As can be appreciated, there can be modifications to the structures and functions described above, as additional embodiments of the invention. The inlet housing 68 and outlet housing 74 can be disposed coaxially or in offset alignment, or on the same end of body 62. In addition, the first and second housings can depend from the same end of the body, where the other end of the body is closed. As well, duckbill valves 63 and 86 can be replaced with any suitable fluid flow control means for one-way flow of fluid materials or gas from a medical accessory into the hollow interior 88 of body 60 or from the interior 88 to the environment. Umbrella valve 124 can be replaced with any suitable fluid flow control means for one-way flow of fluid materials or gas from the medical accessory into the hollow interior 122 of main body 116. In addition, body 62 can be formed integrally, as well as inlet housing 68, outlet housing 74, and gas outlet housing 84 being formed as parts that attach to body 62. Further, gas outlet housing 84 can depend on either end of body 62. Although bayonet slots 80 and 120 are described for retaining the medical accessory with outlet housings 74 and 108, any suitable retaining means can be used. In addition, one bayonet slot 80 and 120 can be used as retaining means. As can be appreciated, the fluid flow control means can be actuated by the fluid flow to and from a medical accessory, or can be actuated by the medical accessories themselves.

1. An apparatus for controlled flow of fluid materials for use with medical accessories, comprising:
a first housing having a body, a first open end, a second open end, and a hollow interior;
said second end being dimensioned for fit with a first medical accessory for flow of fluid materials from said first medical accessory;
a fluid flow control means disposed within said first housing for controlling the flow of said fluid materials through said hollow interior from said first medical accessory;
a second housing having an open end, a first open end, a hollow interior, and a second open end having an axial bore coaxial with said hollow interior of said first housing;
said second end of said second housing being dimensioned for fit with said first open end of said first housing; and
said first open end of said second housing being dimensioned for fit with a second medical accessory, whereby said second medical accessory receives fluid materials from said first medical accessory by way of the hollow interior of said body.

2. The apparatus according to claim 1, wherein the fluid flow control means comprises a one-way valve to permit controlled flow from said first medical accessory.

3. The apparatus according to claim 2, wherein said one-way valve is an umbrella valve.

4. The apparatus according to claim 1 wherein the fluid flow control means comprises a valve to permit controlled flow to and from said first and second medical accessories.

5. The apparatus according to claim 4 wherein the said valve is a combination valve having a duckbill valve portion at one end of said combination valve, an umbrella valve portion having an aperture at the other end of said combination valve, and a passageway extending from the interior of said duckbill valve portion through the interior of said umbrella valve portion, said passageway being coaxial and co-terminus with said aperture.

6. The apparatus according to claim 4 wherein the said valve comprises at least two one-way valves, where at least one one-way valve permits fluid flow in an opposite direction from at least one other one-way valve.

7. The apparatus according to of claim 1 wherein said fluid flow control means is actuable by said second medical accessory.

8. The apparatus according to claim 5 wherein said second end of said first housing further comprises:
a first circumferential side wall;
a circumferential bottom shoulder attached thereto; a second circumferential side wall depending from said shoulder;
said second circumferential side wall having an inner diameter smaller than the inner diameter of said shoulder and defining an axial bore; and
the end of said second circumferential side wall distal from said bottom shoulder being dimensioned for fit with said first medical accessory.

9. The apparatus according to claim 8 wherein said second housing further includes a circumferential side wall, a bottom plate attached thereto, said bottom plate having an aperture, and a hollow needle disposed within said aperture for fluid communication between said needle and said fluid flow control means.

10. The apparatus according to claim 8 wherein the said ends dimensioned for fit with said medical accessories are luer fittings.

11. The apparatus according to claim 8 wherein the first housing and second housing are formed integrally.

12. The apparatus according to any of claims 1, 2, or 4, wherein the second open end of the first housing comprises:
a body having a hollow interior;
said body having an diameter smaller than the body of the first housing;
said body being removably attached to said body of said first housing;
and said hollow interior being coaxial with said hollow interior of said first housing.

13. The apparatus according to claim 12 wherein the said second open end is affixed to said body of said first housing.

14. The apparatus according to claim 12 wherein the said second housing further comprises:
a circumferential wall extending axially outwardly from said second end of said second housing distal to the first housing;
a shoulder depending radially inwardly from the second end of said second housing distal to said first housing and defining an aperture; and
said aperture being coaxial with said hollow interior of said first housing.

15. The apparatus according to claim 14 wherein the said second housing further includes an actuating means that actuates said second medical accessory for fluid communication between said second medical accessory and said hollow interior of said first housing.

16. The apparatus according to claim 14 wherein said second housing further includes retaining means to maintain said second medical accessory in association with said second housing.

17. The apparatus according to claim 14 wherein second housing further includes retaining means to maintain said second medical accessory in association with said second housing, wherein said retaining means comprise at least one bayonet slot.

18. The apparatus according to claim 14 wherein second housing further includes retaining means to maintain said second medical accessory in association with said second housing, wherein said retaining means comprise at least one bayonet slot and at least one bead associated with said bayonet slot.

19. The apparatus according to claim 14 wherein any of the second open end of said first housing, said first housing, and said second housing are integrally formed in any combination.

20. The apparatus according to claim 14 wherein said shoulder further includes an upstanding annular ring to assist in creating a fluid tight fit with said second medical accessory.

21. The apparatus according to any of claims 1, 2, or 3 wherein:
the second open end of the first housing comprises a body having a hollow interior, said body having an diameter smaller than the body of the first housing, said body being removably attached to said body of said first housing; and said hollow interior being coaxial with said hollow interior of said first housing;
said second open end is affixed to said body of said first housing;
said second housing further comprising a circumferential wall extending axially outwardly from said second end of said second housing distal to the first housing, a shoulder depending radially inwardly from the second end of said second housing distal to said first housing and defining an aperture where said aperture is coaxial with said hollow interior of said first housing;
said second housing further including an actuating means that acutates said second medical accessory for fluid communication between said second medical accessory and said hollow interior of said first housing;
said second housing further including retaining means in the form of at least one bayonet slot and at least one bead associated with said bayonet slot to maintain said second medical accessory in association with said second housing; and
said shoulder further including an upstanding annular ring to assist in creating a fluid tight fit with said second medical accessory.

22. A controlled flow apparatus for fluid materials for use with medical accessories, comprising:
a body having a hollow interior and two ends:
a first housing depending from one end of said body and in fluid communication with said hollow interior;
the end of said first housing distal to said body being dimensioned for fit with a first medical accessory;
a first fluid flow control means positioned within said first housing to control fluid flow from said first medical accessory through said first housing and into said hollow interior;
a second housing depending from the other end of said body and in fluid communication with said hollow interior;
wherein the end of said second housing distal to said body is dimensioned for fit with a second medical accessory; and
a second fluid flow control means positioned within said second housing for controlled flow of fluid materials from said hollow interior to said second accessory.

23. A controlled flow apparatus according to claim 22 wherein said first fluid flow control means comprises a one way valve to control flow of fluid materials from said first medical accessory to said hollow interior.

24. A controlled flow apparatus according to claim 23 wherein said one way valve is a duckbill valve.

25. A controlled flow apparatus according to claim 22 wherein said first fluid flow means controls flow of fluid materials to and from said first medical accessory.

26. A controlled flow apparatus according to claim 25 wherein said first fluid flow control means is a valve.

27. A controlled flow apparatus according to claim 26 wherein said valve is a combination valve having a duckbill valve portion at one end of said combination valve, an umbrella valve portion having an aperture at the other end of said combination valve, and a passageway extending from the interior of said duckbill valve portion through the interior of said umbrella valve portion, said passageway being coaxial and co-terminus with said aperture.

28. A controlled flow apparatus according to claim 26 wherein the said valve comprises at least two one-way valves, where at least one one-way valve permits fluid flow in an opposite direction from at least one other one-way valve.

29. A controlled flow apparatus according to any of claims 22 or 25 wherein said second fluid flow control means controls flow of fluid materials to and from said hollow interior and said second accessory.

30. A controlled flow apparatus according to claim 29 wherein said second fluid flow control means is a valve.

31. A controlled flow apparatus according to claim 29 wherein said second fluid flow control means is a a combination valve having a duckbill valve portion at one end of said combination valve, an umbrella valve portion having an aperture at the other end of said combination valve, and a passageway extending from the interior of said duckbill valve portion through the interior of said umbrella valve portion, said passageway being coaxial and co-terminus with said aperture.

32. A controlled flow apparatus according to claim 29 wherein the said second fluid flow control means comprises at least two one-way valves, where at least one one-way valve permits fluid flow in an opposite direction from at least one other one-way valve.

33. A controlled flow apparatus according to claim 29 wherein said body comprises two parts joined together.

34. A controlled flow apparatus according to claim 29 wherein said first housing and said second housing are in coaxial alignment.

35. A controlled flow apparatus according to claim 29 wherein said first housing and said second housing depend from the same end of said body.

36. A controlled flow apparatus according to claim 29 wherein said first housing and said second housing are in offset alignment.

37. A controlled flow apparatus according to claim 29 further including a third housing depending from an end of said body, said third housing being in fluid communication with said hollow interior, and a gas flow control means positioned within said third housing to control the expression of gaseous material from said hollow interior to the environment without permitting transfer of fluid materials from said hollow interior to the environment.

38. A controlled flow apparatus according to claim 29 further including a third housing depending from an end of said body, said third housing being in fluid communication with said hollow interior, and a gas flow control means positioned within said third housing to control the expression of gaseous material from said hollow interior to the environment without permitting transfer of fluid materials from said hollow interior to the environment, and wherein a hydrophobic membrane is positioned in association with the aperture of said third housing proximal to said hollow interior to prevent flow of fluid materials from said hollow interior to the environment through said third housing.

39. A controlled flow apparatus according to claim 29 further including a third housing depending from an end of said body, said third housing being in fluid communication with said hollow interior, and a gas flow control means positioned within said third housing to control the expression of gaseous material from said hollow interior to the environment without permitting transfer of fluid materials from said hollow interior to the environment, wherein a hydrophobic membrane is positioned in association with the aperture of said third housing proximal to said hollow interior to prevent flow of fluid materials from said hollow interior to the environment through said third housing and wherein an oleophobic membrane is associated with said hydrophobic membrane proximal to said hollow interior.

40. A controlled flow apparatus according to claim 29 further including a third housing depending from an end of said body, said third housing being in fluid communication with said hollow interior, and a gas flow control means positioned within said third housing to control the expression of gaseous material from said hollow interior to the environment without permitting transfer of fluid materials from said hollow interior to the environment, wherein said gas flow control means comprises a one-way valve.

41. A controlled flow apparatus according to claim 40 wherein the one-way valve is a duckbill valve.

42. A controlled flow apparatus according to claim 29, wherein the said second housing further comprises a circumferential wall extending axially outwardly from said body, a shoulder depending radially inwardly from the end of said second housing proximal to said body and defining an aperture, and the end of said second fluid flow control means distal to said interior being biased into fluid tight engagement with said shoulder.

43. A controlled flow apparatus according to claim 29 wherein the said second housing further comprises a circumferential wall extending axially outwardly from said body, a shoulder depending radially inwardly from the end of said second housing proximal to said body and defining an aperture, and the end of said second fluid flow control means distal to said interior being biased into fluid tight engagement with said shoulder, and wherein said second fluid control means includes a valve head and further includes a circumferential valve seat with an aperture attached to said shoulder, wherein said valve head is biased against said valve seat for fluid tight fit.

44. A controlled flow apparatus according to claim 29 wherein the said second housing further comprises a circumferential wall extending axially outwardly from said body, a shoulder depending radially inwardly from the end of said second housing proximal to said body and defining an aperture, and the end of said second fluid flow control means distal to said interior being biased into fluid tight engagement with said shoulder, and wherein said second fluid control means includes a valve head and further includes a circumferential valve seat with an aperture attached to said shoulder, wherein said valve head is biased against said valve seat for fluid tight fit and said second fluid control means further comprises a nose portion which extends axially outwardly from the end of said housing proximal to said body.

45. A controlled flow apparatus according to claim 29 wherein the said second housing further comprises a circumferential wall extending axially outwardly from said body, a shoulder depending radially inwardly from the end of said second housing proximal to said body and defining an aperture, and the end of said second fluid flow control means distal to said interior being biased into fluid tight engagement with said shoulder, and wherein said second fluid control means includes a valve head and further includes a circumferential valve seat with an aperture attached to said shoulder, wherein said valve head is biased against said valve seat for fluid tight fit, and wherein said valve seat is made of elastomeric material.

46. A controlled flow apparatus according to claim 29 wherein the said second housing further comprises a circumferential wall extending axially outwardly from said body, a shoulder depending radially inwardly from the end of said second housing proximal to said body and defining an aperture, and the end of said second fluid flow control means distal to said interior being biased into fluid tight engagement with said shoulder, and wherein said second fluid control means includes a valve head and further includes a circumferential valve seat with an aperture attached to said shoulder, wherein said valve head is biased against said valve seat for fluid tight fit, and wherein said valve seat further includes an upstanding annular ring to assist in creating a fluid tight fit with said second medical accessory.

47. A controlled flow apparatus according to claim 29 wherein said second housing further includes retaining means to maintain said second medical accessory in association with said second housing.

48. A controlled flow apparatus according to claim 29 wherein said second housing further includes retaining means to maintain said second medical accessory in association with said second housing, and wherein the retaining means comprise at least one bayonet slot.

49. A controlled flow apparatus according to claim 29 wherein the said second fluid flow control means is actuable by said second medical accessory.

50. A controlled flow apparatus according to claim 29 wherein said first, second, and third housings are formed separately from, and are attached to, said body.

51. A controlled flow apparatus according to claim 29, further including:
said first fluid flow control comprising a one way valve to control flow of fluid materials from said first medical accessory to said hollow interior;
a third housing depending from an end of said body, said third housing being in fluid communication with said hollow interior, and a gas flow control means positioned within said third housing to control the expression of gaseous material from said hollow interior to the environment without permitting transfer of fluid materials from said hollow interior to the environment;
a hydrophobic membrane positioned in association with the aperture of said third housing proximal to said hollow interior to prevent flow of fluid materials from said hollow interior to the environment through said third housing;
an oleophobic membrane associated with said hydrophobic membrane proximal to said hollow interior;
said gas flow control means comprising a one-way valve;
said second housing further comprising a circumferential wall extending axially outwardly from said body, a shoulder depending radially inwardly from the end of said second housing proximal to said body and defining an aperture, and the end of said second fluid flow control means distal to said interior being biased into fluid tight engagement with said shoulder;
said second fluid control means further including a valve head and further includes a circumferential valve seat with an aperture attached to said shoulder, wherein said valve head is biased against said valve seat for fluid tight fit and a nose portion which extends axially outwardly from the end of said housing proximal to said body;
said valve seat being made of elastomeric material and further including an upstanding annular ring to assist in creating a fluid tight fit with said second medical accessory; and
said second housing further including at least one bayonet slot to maintain said second medical accessory in association with said second housing.

52. A controlled flow apparatus according to claim 42 wherein said second housing further includes retaining means to maintain said second medical accessory in association with said second housing.

53. A controlled flow apparatus according to claim 42 wherein said second housing further includes retaining means to maintain said second medical accessory in association with said second housing, and wherein the retaining means comprise at least one bayonet slot.
